(19) United States
(12) Patent Application Publication
(10) Pub. No.: US 2024/0254408 A1
(43) Pub. Date: Aug. 1, 2024

(54) MIXTURE OF MONOBRANCHED AND POLYBRANCHED FATTY ACIDS

(71) Applicant: Katholieke Universiteit Leuven, Leuven (BE)

(72) Inventors: Dorien Kerstens, Lier (BE); Bert Sels, Westerlo (BE)

(21) Appl. No.: 18/564,505

(22) PCT Filed: May 27, 2022
(86) PCT No.: PCT/EP2022/064451

§ 371 Date: Nov. 27, 2023

(30) Foreign Application Priority Data
May 27, 2021 (EP) 21176339.6

(51) Int. Cl. C11C 3/12; B01J 29/70; B01J 35/64

(52) U.S. Cl. CPC C11C 3/126; B01J 29/7042; B01J 29/7046; B01J 35/643

(57) ABSTRACT

A composition of branched fatty acids or esters thereof and the processes for preparing such compositions and to a process of producing a composition of branched C10-C24 fatty acids or esters thereof with a high portion, at least 70% by weight, of mono and polybranched C10-C24 fatty acids or esters thereof.

### CROSS-REFERENCE TO RELATED APPLICATIONS

This application is a national phase entry under 35 U.S.C. § 371 of International Patent Application PCT/EP2022/064451, filed May 27, 2022, designating the United States of America and published as International Patent Publication WO 2022/248688 A1 on Dec. 1, 2022, which claims the benefit under Article 8 of the Patent Cooperation Treaty of European Patent Application Serial No. 21176339.6, filed May 27, 2021.

### TECHNICAL FIELD

The disclosure relates to a composition of branched fatty acids or esters thereof and the processes for preparing such compositions and to a process of producing a composition of branched C10-C24 fatty acids or esters thereof with a high portion, at least 70% by weight, of mono and polybranched C10-C24 fatty acids or esters thereof.

### BACKGROUND

More particularly, the disclosure relates to a composition of branched C₁₀-C₂₄ fatty acids or esters thereof, comprising at least 70% by weight of mono and polybranched C10-C24 fatty acids or esters thereof and a ratio of monobranched/polybranched C₁₀-C₂₄ fatty acids or esters thereof smaller than 5:1 by weight based on the total weight of the composition.

Several documents are cited throughout the text of this specification. Each of the documents herein (including any manufacturer's specifications, instructions etc.) are hereby incorporated by reference; however, there is no admission that any document cited is indeed prior art of the disclosure.

### DESCRIPTION OF THE RELATED ART

Presently, branched fatty acids are produced industrially as a by-product of the thermal polymerization of unsaturated fatty acids or fatty acid esters with acid clays as a catalyst. After reaction, a product consisting of a polymeric and a monomeric fraction, is obtained. The polymeric fraction mainly consists of dimers and trimers, while the branched fatty acids can be found in the monomeric fraction. As multiple reactions, e.g., cis trans isomerization, branching, aromatization, double bond shift, hydrogen transfer, occur at the same time, the current reaction product is extremely complex. (1-3)

Current mixtures after processing have monomer fractions that usually vary around 35 wt % as the catalyst primarily forms oligomeric compounds. Multiple purification steps like crystallization and distillation are thus necessary in order to obtain a product consisting primarily of branched fatty acids, which can be considered as a major disadvantage of this process. (4) Since the monomer fraction comprises around 50 wt % branched fatty acids, the overall maximum yield of the process for branched fatty acids is around 17.5 wt %, with the classic process. (5)

Research on the isomerization of unsaturated fatty acids to branched fatty acids has been published in multiple patents and scientific journals. Despite the fact that the use of clay catalysts for the production of branched fatty acids opposes multiple disadvantages, early patents still use clays like Montmorillonite and Bentonite for the isomerization of fatty acids. In these patents, the use of co-catalysts like dichloromethane and activated carbon is described, which should cause a higher yield of branched fatty acids. Despite this, Neuss et al. report mixtures with only 40%, while Foglia et al. report a product with only 57 wt % of branched fatty acids. Moreover, the function of these co-catalysts remains unclear. (6,7)

As the obtained yields of branched fatty acids remain low, researchers have been looking for new catalysts in order to increase the obtained yields. Commercial zeolites are proposed as promising catalysts for the isomerization of unsaturated fatty acids to branched fatty acids. The structure of the zeolite makes it possible to obtain higher yields of branched fatty acids as the pores are too small to form oligomeric side products, but large enough to make diffusion of the branched product possible. Besides this, zeolites have shown to be reusable for multiple times. (8,9)

The oldest patents using zeolites mainly focus on Mordenite and other one dimensional zeolites. It is in one of these patents that the use of water or a small alcohol as an additive is mentioned for the first time. There is assumed that adding water, when the substrate is a fatty acid, or a lower alcohol, when the substrate is a fatty acid ester, suppresses the formation of acid anhydrides by dehydration or dealcoholation of the reagent. (8) However, the use of water or a lower alcohol is countered by another patent published one year later. In this patent, a 68% yield is reached with less catalyst, a lower reaction temperature, in a shorter time period and without adding water. (9)

A few years later, the attention shifts toward other zeolites like Beta. With H-Beta, it is possible to reach conversions up to 74% and to have a product consisting of 46% branched fatty acids. (12)

There is a need in the art for an improved process of producing branched fatty acids, particularly a mixture of monobranched and polybranched fatty acids.

### BRIEF SUMMARY

A process has now been discovered for producing branched C₁₀-C₂₄ fatty acids with at least 70% by weight of mono and polybranched C10-C24 fatty acids or esters thereof and a ratio of monobranched/polybranched C₁₀-C₂₄ fatty acids or esters thereof smaller than 5:1 by weight based on the total weight of the composition by heating a starting material comprising at least 80% by weight of linear monoethylenically unsaturated C₁₀-C₂₄ fatty acid(s) in the presence of a microporous aluminosilicate catalyst and no other additives. No additives such as dichloromethane, activated carbon, water or light alcohols (methanol, ethanol), Lewis base (for instance, triphenylphosphine, triethylenediamine, a combination of triphenylphosphine and triethylenediamine or metalloaluminophosphate molecular sieves had to be used).

The disclosure relates to a composition of branched fatty acids or esters thereof and the processes for preparing such compositions. More particularly, the disclosure relates to a composition of branched C₁₀-C₂₄ fatty acids or esters thereof, comprising at least 70% by weight of mono and polybranched C₁₀-C₂₄ fatty acids or esters thereof and a ratio of monobranched/polybranched C₁₀-C₂₄ fatty acids or esters thereof smaller than 5:1 by weight based on the total weight of the composition.

The disclosure provides a way to obtain a composition of branched C₁₀-C₂₄ fatty acids obtained after a process with a starting material comprising at least 80% by weight of linear monoethylenically unsaturated C₁₀-C₂₄ fatty acid(s) based on the total weight of the starting material, isomerizing the linear monoethylenically unsaturated C₁₀-C₂₄ fatty acid(s) from the starting material, by heating in the presence of an orthorhombic 10-membered-ring pore one-dimensional straight channel zeolite isomerization catalyst.

The disclosure provides a way to obtain a composition of branched C₁₀-C₂₄ fatty acids obtained after a process with a starting material comprising at least 80% by weight of linear monoethylenically unsaturated C₁₀-C₂₄ fatty acid(s) based on the total weight of the starting material, isomerizing the linear monoethylenically unsaturated C₁₀-C₂₄ fatty acid(s) from the starting material, by heating in the presence of an orthorhombic 10-membered-ring pore one-dimensional straight channel zeolite isomerization catalyst as sole catalysts.

The disclosure provides a way to obtain a composition of branched C₁₀-C₂₄ fatty acids obtained after a process with a starting material comprising at least 80% by weight of linear monoethylenically unsaturated C₁₀-C₂₄ fatty acid(s) based on the total weight of the starting material, isomerizing the linear monoethylenically unsaturated C₁₀-C₂₄ fatty acid(s) from the starting material, by heating in the presence of a single zeolite catalyst of the group of the orthorhombic 10-membered-ring pore one-dimensional straight channel zeolite isomerization catalysts.

The disclosure provides a way to obtain a composition of branched C₁₀-C₂₄ fatty acids obtained after a process with a starting material comprising at least 80% by weight of linear monoethylenically unsaturated C₁₀-C₂₄ fatty acid(s) based on the total weight of the starting material, isomerizing the linear monoethylenically unsaturated C₁₀-C₂₄ fatty acid(s) from the starting material, by heating in the presence of an orthorhombic 10-membered-ring pore one-dimensional straight channel zeolite isomerization catalyst without a co catalyst.

The disclosure provides a way to obtain a composition of branched C₁₀-C₂₄ fatty acids obtained after a process with a starting material comprising at least 80% by weight of linear monoethylenically unsaturated C₁₀-C₂₄ fatty acid(s) based on the total weight of the starting material, isomerizing the linear monoethylenically unsaturated C₁₀-C₂₄ fatty acid(s) from the starting material, by heating in the presence of an orthorhombic 10-membered-ring pore one-dimensional straight channel zeolite isomerization catalyst without an additional catalyst selected of the group consisting of 1) dichloromethane, 2) activated carbon, 3) any additives like water or light alcohols (methanol, ethanol), 4) a Lewis base catalyst such as the Lewis base catalyst triphenylphosphine, 5) the Lewis base catalyst triethylenediamine and combinations thereon.

The disclosure also provides a way to obtain a composition of branched C₁₀-C₂₄ fatty acids obtained after a process with a starting material comprising at least 80% by weight of linear monoethylenically unsaturated C₁₀-C₂₄ fatty acid(s) based on the total weight of the starting material, isomerizing the linear monoethylenically unsaturated C₁₀-C₂₄ fatty acid(s) from the starting material, by heating in the presence of an orthorhombic (high silica) 10-membered ring (10MR) zeolite composed of 5-, 6-, and 10-rings whereby 10-ring channels (with 10-membered ring openings) are linear unidirectional and one-dimensional (noninterconnecting).

The disclosure also provides a way to obtain a composition of branched C10-C24 fatty acids obtained after a process with a starting material comprising at least 80% by weight of linear monoethylenically unsaturated C10-C24 fatty acid(s) based on the total weight of the starting material, isomerizing the linear monoethylenically unsaturated C10-C24 fatty acid(s) from the starting material, by heating in the presence of an orthorhombic (high silica) 10-membered ring (10MR) zeolite composed of 5-, 6-, and 10-rings whereby 10-ring channels (with 10-membered ring openings) are linear unidirectional and one-dimensional non-interconnecting and in the absence of any additives.

Such zeolite catalyst does not contain interconnecting channels with otherwise would have large intersection spaces of more than 6.2 Å. such as in ZSM-35 and ZSM-5. ZSM-35 has ferrierite topology and a two-dimensional textural structure and a 10-membered ring channel of 5.4-4.2 Å, perpendicular to the 8-membered ring channel of 4.8-3.5 Å. The intersection of the two-dimensional channels provides a large space, and the maximum diameter of a sphere that can be included is 6.31 Å and thus larger than that in ZSM-22 and ZSM-23. ZSM-5 has a three-dimensional crosslinking network structure. Perpendicular to the plane of the two-dimensional 10-membered ring sinusoidal channel, there is another straight, 10-membered ring channel passing through the plane and intercrossing with the sinusoidal channel. The intersections of these 10-membered ring channels, with sizes of 5.6 Å-5.3 Å and 5.5 Å-5.1 Å, respectively, and are slightly larger than those in ZSM-35 and the maximum diameter of a sphere that can be included is 6.36 Å and thus much larger than larger than that in ZSM-22 and ZSM-23.

The one dimensional straight channel zeolite ZSM-22 and ZSM-23 are suitable isomerization catalyst for the process of manufacturing the composition of disclosure, while ZSM-35 and ZSM-5 were demonstrated to be unsuitable for the process of manufacturing the composition of disclosure. ZSM-22 and ZSM-23 is a one dimensional straight channel zeolite, while ZSM-5 is a three dimensional channel zeolite containing two types of interconnecting channels: straight channels (5.6-5.3 Å) and sinusoidal channels (5.5-5.1 A), which can provide a wider space than the space of the straight channels of the 10-membered rings in ZSM-22.

A particularly suitable isomerization catalyst for disclosure is a zeolite of the group of a ZSM-22 zeolite with TON topology, ZSM-23 zeolite with MTT topology or a ZSM-23/ZSM-22 with MTT (ZSM-23) and TON (ZSM-22) frameworks.

The disclosure also provides a process for preparing a composition of branched C₁₀-C₂₄ fatty acids or esters thereof, comprising at least 70% by weight of mono and polybranched C₁₀-C₂₄ fatty acids or esters thereof and a ratio of monobranched/polybranched C₁₀₋₂₄ fatty acids or esters thereof smaller than 5:1 by weight based on the total weight of the composition from a starting material comprising at least 80% by weight of linear monoethylenically unsaturated C₁₀-C₂₄ fatty acid(s) based on the total weight of the starting material, whereby this process comprises isomerizing the linear monoethylenically unsaturated C₁₀-C₂₄ fatty acid(s) from the starting material, by heating in the presence of an isomerization catalyst whereby the isomerization catalyst comprises an orthorhombic 10-membered-ring pore one-dimensional straight channel zeolite.

According to the disclosure there is also provided a process for preparing a composition of branched C₁₀-C₂₄ fatty acids according to embodiment 1 from a starting material comprising at least 80% by weight of linear monoethylenically unsaturated C₁₀-C₂₄ fatty acid(s) based on the total weight of the starting material, this process comprising the following step: (i) isomerizing the linear monoethylenically unsaturated C₁₀-C₂₄ fatty acid(s) from the starting material, by heating in the presence of the isomerization catalyst, (ii) separating the monomeric fraction from the oligomeric fraction formed in step (i) and (iii) purifying the monomeric fraction to obtain the composition of branched C₁₀-C₂₄ fatty acids.

Surprisingly, this composition was obtainable by heating the start material in the presence of the isomerization catalyst of disclosure and in the absence of any addition additives whereby the isomerization catalyst. In the above described process of disclosure a composition of branched C₁₀-C₂₄ fatty acids can be prepared from a starting material comprising at least 80% by weight of linear monoethylenically unsaturated C₁₀-C₂₄ fatty acid(s) based on the total weight of the starting material, by a process comprising the steps of (i) isomerizing the linear monoethylenically unsaturated C₁₀-C₂₄ fatty acid(s) from the starting material, by heating in the presence of the isomerization catalyst and in the absence of any additional additives, (ii) separating the monomeric fraction from the oligomeric fraction formed in step (i) and (iii) purifying the monomeric fraction to obtain the composition of branched C₁₀-C₂₄ fatty acids.

A suitable isomerization catalyst is an orthorhombic 10-membered-ring pore one-dimensional straight channel zeolites, without interconnecting channels, for instance, an orthorhombic 10-membered-ring pore one-dimensional straight channel zeolites, without interconnecting channels, which otherwise creates intersection spaces of more than 6.2 Å.

A suitable isomerization catalyst for disclosure is an orthorhombic high silica 10-membered ring (10MR) zeolite composed of 5-, 6-, and 10-rings whereby 10-ring channels (with 10-membered ring openings) are linear unidirectional and one-dimensional (noninterconnecting) and have a pore diameter of 0.44 nm-0.56 nm×0.51 nm-0.59 nm, preferably of 0.45 nm-0.47 nm×0.52 nm-0.58 nm orthorhombic high silica 10-membered ring (10MR) zeolite composed of 5-, 6-, and 10-rings whereby 10-ring channels (with 10-membered ring openings) are linear unidirectional and one-dimensional (noninterconnecting) and have openings that are in the range of 5.5-5.9×4.4-4.7 angstroms and preferably 5.6-5.8×4.5-4.7 angstroms and most preferably about 5.7×4.6 angstroms. Particular suitable isomerization catalyst is a zeolite of the group of a ZSM-22 zeolite with TON topology, ZSM-23 zeolite with MTT topology or a ZSM-23/ZSM-22 with MTT (ZSM-23) and TON (ZSM-22) frameworks and preferably such are not mesoporized.

By using an inventive process, it is possible to produce a composition of branched C₁₀-C₂₄ fatty acids or esters thereof, comprising 1) at least 70% by weight of mono and polybranched C₁₀-C₂₄ fatty acids or esters thereof and 2) a ratio of monobranched/polybranched C₁₀₋₂₄ fatty acids or esters thereof smaller than 5:1 by weight based on the total weight of the composition. And such branched fatty acid alkyl esters and fatty acids products of the disclosure are particularly suitable and can be utilized in a lubricant, a personal care and/or a home care composition. Such lubricant may integrate a base oil and such personal care composition may integrate an active ingredient and/or a pigment or a colorant.

In another aspect, of disclosure provides there is provided a composition of branched C10-C24 fatty acids or esters thereof, comprising or essentially consisting of 1) at least 70% by weight of mono and polybranched C₁₀-C₂₄ fatty acids or esters thereof and 2) a ratio of monobranched/polybranched C₁₀₋₂₄ fatty acids or esters thereof smaller than 5:1 by weight based on the total weight of the composition or from 1.5:1 to 5:1 by weight based on the total weight of the composition. The disclosure also provides the use thereof for processing it in a lubricant, a personal care and/or a home care composition. The disclosure further provides that the amount of monobranched C10-C24 fatty acids or esters thereof in the composition is at least 45% by weight based on the total weight of the composition. The disclosure further provides that the amount of polybranched C₁₀-C₂₄ fatty acids or esters thereof in the composition is ranges from 0.1 to 30% by weight based on the total weight of the composition. In a further aspect of the disclosure, the amount of cyclic fatty acids in the composition ranges from 0.1 to 5% by weight based on the total weight of the composition. In one aspect of the disclosure, the cyclic compounds comprise alicyclic carboxylic acid(s) or ester(s) thereof, which content ranges from 0.1 to 5% by weight based on the total weight of the composition. In yet another aspect of disclosure the amount of linear and branched lactones ranges from 0.1 to 5% by weight based on the total weight of the composition. In yet another aspect of disclosure, the amount of oligomers ranges from 0.1 to 8.5% by weight based on the total weight of the composition.

Within a particular embodiment of the disclosure, the composition has an acid value higher than 165 mg KOH/g.

In a preferred embodiment of disclosure the composition further comprises 1) at least 45% by weight of monobranched C₁₀-C₂₄ fatty acids or esters thereof, 2) 0.1 to 30% by weight of polybranched C₁₀-C₂₄ fatty acids or esters thereof, 3) 0.1 to 5% by weight of cyclic compounds, 6) 0.1 to 5% by weight of linear and branched lactones, 4) 0.1 to 8.5% by weight of oligomers and 5) an acid value higher than 165 mg KOH/g by weight based on the total weight of the composition.

This composition of branched C₁₀-C₂₄ fatty acids of disclosure is obtainable from a starting material comprising at least 80% by weight of linear monoethylenically unsaturated C₁₀-C₂₄ fatty acid(s) based on the total weight of the starting material by a process comprising isomerizing the linear monoethylenically unsaturated C₁₀-C₂₄ fatty acid(s) from the starting material, by heating in the presence of an isomerization catalyst and in the absence of any additives and, in particular, by a process comprising the following step: (i) isomerizing the linear monoethylenically unsaturated C₁₀-C₂₄ fatty acid(s) from the starting material, by heating in the presence of an isomerization catalyst and in the absence of any additives, (ii) separating the monomeric fraction from the oligomeric fraction formed in step (i) and (iii) purifying the monomeric fraction to obtain the composition of branched C₁₀-C₂₄ fatty acids. This process described above may be embodied as that therein an isomerization catalyst comprises an orthorhombic 10-membered-ring pore one-dimensional straight channel zeolite and preferably an isomerization catalyst is an orthorhombic 10-membered-ring pore one-dimensional straight channel zeolites, without interconnecting channels. Particular suitable isomerization catalyst have an orthorhombic 10-membered-ring pore one-dimensional straight channel zeolites, without interconnecting channels, which otherwise creates intersection spaces of more than 6.2 Å, for instance, such isomerization catalyst is an orthorhombic high silica 10-membered ring (10MR) zeolite composed of 5-, 6-, and 10-rings whereby 10-ring channels (with 10-membered ring openings) are linear unidirectional and one-dimensional (non interconnecting) and have a pore diameter of 0.44 nm-0.56 nm×0.51 nm-0.59 nm, preferably of 0.45 nm-0.47 nm×0.52 nm-0.58 nm.

As demonstrated by the examples the process to make the composition of present advantageously comprises isomerization catalyst is a zeolite of the group of a ZSM-22 zeolite with TON topology, ZSM-23 zeolite with MTT topology or a ZSM-23/ZSM-22 with MTT (ZSM-23) and TON (ZSM-22) frameworks.

Further scope of applicability of the disclosure will become apparent from the detailed description given hereinafter. However, it should be understood that the detailed description and specific examples, while indicating preferred embodiments of the disclosure, are given by way of illustration only, since various changes and modifications within the spirit and scope of the disclosure will become apparent to those skilled in the art from this detailed description. It is to be understood that both the foregoing general description and the following detailed description are exemplary and explanatory only and are not restrictive of the disclosure, as claimed.

Herein described aspects and preferred embodiments of the disclosure may be presented/described.

1. A composition comprising branched C10-C24 fatty acids with 1) at least 70% by weight of mono and polybranched C₁₀-C₂₄ fatty acids or esters thereof and 2) a ratio of monobranched/polybranched C₁₀-C₂₄ fatty acids or esters thereof smaller than 5:1 by weight based on the total weight of the composition, which is a reaction product of a starting material comprising at least 80% by weight of linear monoethylenically unsaturated C₁₀-C₂₄ fatty acid(s), based on the total weight of the starting material, heated in the presence of a microporous aluminosilicate catalyst and in the absence of a Lewis base.

2. The composition according to embodiment 1, characterized in that the catalyst has 10-ring linear channels without intersecting channels.

3. The composition according to embodiment 1, characterized in that the catalyst has 10-ring linear channels without interconnecting channels, which otherwise creates intersection spaces of more than 6.2 angstroms.

4. The composition according to any one of the embodiments 1 to 3, characterized in that the catalyst is an orthorhombic 10-membered ring (10MR) zeolite composed of 5-, 6-, and 10-rings whereby 10-ring channels (with 10-membered ring openings) are linear unidirectional and one-dimensional.

5. The composition according to any one of the embodiments 1 to 4, characterized in that the catalyst has pore/channel openings under 7.5 angstroms.

6. The composition according to any one of the embodiments 1 to 5, characterized in that the 10-ring linear channels of the catalyst have a pore diameter of 0.44 nm-0.56 nm×0.51 nm-0.59 nm, preferably of 0.45 nm-0.47 nm×0.52 nm-0.58 nm.

7. The composition according to any one of the embodiments 1 to 5, characterized in that the 10-ring linear channels of the catalyst have openings that are in the range of 5.5-5.9×4.4-4.7 angstroms and preferably 5.6-5.8×4.5-4.7 angstroms and most preferably about 5.7×4.6 angstroms.

8. The composition according to any one of the embodiments 1 to 5, characterized in that the catalyst is a zeolite of the group of a ZSM-22 zeolite with TON topology, ZSM-23 zeolite with MTT topology or a ZSM-23/ZSM-22 with MTT (ZSM-23) and TON (ZSM-22) frameworks.

9. The composition according to any one of the embodiments 1 to 8, whereby the starting material is heated in the absence of a catalyst having a mesoporous crystalline phase.

10. The composition according to any one of the embodiments 1 to 9, whereby the starting material is heated in the absence of a metal containing catalyst.

11. The composition according to any one of the embodiments 1 to 9, whereby the starting material is heated in the absence of a catalyst containing transition metals, post transition metals, Ln series elements, or an element of the group consisting of B, Ti, Ga, Zr, Ge, Va, Cr, Sb, Nb, and Y.

12. The composition according to any one of the embodiments 1 to 10, whereby the starting material is heated in the absence of an additive or catalyst selected of the group consisting of dichloromethane, activated carbon, water or light alcohols (methanol, ethanol), the Lewis base catalyst triphenylphosphine, the Lewis base catalyst triethylenediamine, a combination of Lewis base catalyst triphenylphosphine, the Lewis base catalyst triethylenediamine and metalloaluminophosphate molecular sieves.

13. The composition according to any one of the embodiments 1 to 12, which is the reaction product of (i) isomerizing the linear monoethylenically unsaturated C₁₀-C₂₄ fatty acid(s) from the starting material, by heating in the presence of the catalyst (ii) separating the monomeric fraction from the oligomeric fraction formed in step (i) and (iii) purifying the monomeric fraction to obtain the composition of branched C₁₀-C₂₄ fatty acids.

14. The composition according to any one of the embodiments 1 to 13, characterized in that it is a composition of branched C₁₀-C₂₄ fatty acids or esters thereof, comprising 1) at least 70% by weight of mono and polybranched C₁₀-C₂₄ fatty acids or esters thereof and 2) a ratio of monobranched/polybranched C₁₀₋₂₄ fatty acids or esters thereof smaller than 5:1 by weight based on the total weight of the composition.

15. The composition according to any one of embodiments 1 to 14 wherein the ratio monobranched/polybranched C₁₀-C₂₄ fatty acids or esters thereof ranges from 1.5:1 to 5:1 by weight based on the total weight of the composition.

16. The composition according any one of embodiments 1 to 15, wherein the amount of monobranched C₁₀-C₂₄ fatty acids or esters thereof is at least 45% by weight based on the total weight of the composition.

17. The composition according to any of embodiments 1 to 16, wherein the amount of polybranched C₁₀-C₂₄ fatty acids or esters thereof ranges from 0.1 to 30% by weight based on the total weight of the composition.

18. The composition according to any one of the embodiments 1 to 17, wherein the amount of cyclic fatty acids ranges from 0.1 to 5% by weight based on the total weight of the composition.

19. The composition according to any one of the embodiments 1 to 18, wherein cyclic compounds comprise alicyclic carboxylic acid(s) or ester(s) thereof, which content ranges from 0.1 to 5% by weight based on the total weight of the composition.

20. The composition according to any one of the embodiments 1 to 19, wherein the amount of linear and branched lactones ranges from 0.1 to 5% by weight based on the total weight of the composition.

21. The composition according to any one of the embodiments 1 to 20, wherein the amount of oligomers ranges from 0.1 to 8.5% by weight based on the total weight of the composition.

22. The composition according to any one of the embodiments 1 to 21, wherein the acid value is higher than 165 mg KOH/g.

23. The composition according to any one of the embodiments 1 to 22, further comprising 1) at least 45% by weight of monobranched C₁₀-C₂₄ fatty acids or esters thereof, 2) 0.1 to 30% by weight of polybranched C₁₀-C₂₄ fatty acids or esters thereof, 3) 0.1 to 5% by weight of cyclic compounds, 6) 0.1 to 5% by weight of linear and branched lactones, 4) 0.1 to 8.5% by weight of oligomers and 5) an acid value higher than 165 mg KOH/g by weight based on the total weight of the composition.

### DETAILED DESCRIPTION

The following detailed description of the disclosure refers to the accompanying drawings. The same reference numbers in different drawings identify the same or similar elements. Also, the following detailed description does not limit the disclosure. Instead, the scope of the disclosure is defined by the accompanying claims and equivalents thereof.

A mesoporous pore range is generally in the range of from 13 to 200 Angstroms and the microporous pore range of the pore/channel openings of typical zeolites ranges from 3-7.5 angstroms.

By “branched” fatty acid, it is intended that the hydrocarbon chain of the monocarboxylic fatty acid bears one or more alkyl side group(s), which is/are generally short.

The following detailed description of the disclosure refers to the accompanying drawings. The same reference numbers in different drawings identify the same or similar elements. Also, the following detailed description does not limit the disclosure. Instead, the scope of the disclosure is defined by the accompanying claims and equivalents thereof.

A mesoporous pore range is generally in the range of from 13 to 200 Angstroms and the microporous pore range of the pore/channel openings of typical zeolites ranges from 3-7.5 angstroms.

By “branched” fatty acid, it is intended that the hydrocarbon chain of the monocarboxylic fatty acid bears one or more alkyl side group(s), which is/are generally short.

By “short alkyl side group”, it is intended a group comprising less than 5 carbon atoms. More particularly, each short alkyl side group is linear and still more particularly, is chosen among the group constituted by methyl, ethyl and propyl. Preferably each short alkyl side group is a methyl and/or an ethyl, more preferably a methyl.

By “branched C10-C24 fatty acids or esters thereof”, it is then intended polybranched C10-C24 fatty acids or esters of polybranched C10-C24 fatty acids, and optionally monobranched C10-C24 fatty acids or esters of monobranched C10-C24 fatty acids, respectively.

By “monobranched” fatty acid, it is intended that the linear hydrocarbon chain of the fatty acid bears only one alkyl side group, which is generally short.

By “polybranched” fatty acid, it is intended that the linear hydrocarbon chain of the fatty acid bears two or more alkyl side groups, which are generally short.

“Cyclic compounds” include but are not limited to alicyclic carboxylic acids or esters thereof, aromatic(s), alkylcyclopentanone(s) and mixture thereof.

ZSM-22 and ZSM-23 zeolites suitable for the disclosure are of the class of one-dimensional zeolites and more particularly one-dimensional straight channel zeolites, yet more particularly of the type of the orthorhombic 10-membered-ring pore one-dimensional straight channel zeolites, without interconnecting channels.

ZSM-23, a high-silica zeolite, is orthorhombic, space group, Pmmn, with lattice parameters of: a=5.01±0.02 Å, b=21.52±0.04 Å, and c=11.13±0.03 Å. The crystal structures of ZSM-22 and ZSM-23 are closely related in that both zeolites contain structurally identical subunits, which generate non interpenetrating, one-dimensional channels defined by 10-rings, which are parallel to the short 5 Å axis. The 10-ring channel dimensions in ZSM-22 and ZSM-23 are essentially the same, though subtle differences exist in the shapes of the openings. The framework topology of this zeolite is composed of 5-, 6-, and 10-rings without intersecting channels, and that 10-ring linear channels have a pore diameter of 0.45 nm×0.52 nm.

ZSM-22, an orthorhombic high silica zeolite (Cmcm, a=13.86±0.03 Å, b=17.41±0.04 Å, and c=5.04±0.02 Å), has a framework consisting of 5-, 6- and 10-rings. The structure contains ferrierite sheets of the type previously found in ZSM-5, ZSM-11 and ZSM-35 and sheets of 6-rings similar to those of the rare zeolite bikitaite. The channel system is linear unidirectional and one-dimensional (noninterconnecting) with 10-membered ring openings that are in the range of 5.5-5.9×4.4-4.7 Å and preferably 5.6-5.8×4.5-4.7 Å and most preferably about 5.7×4.6 Å. The 10-ring channels are smaller than those found previously in ZSM-5, ZSM-11 and ZSM-35.

An example of additives are co-catalysts like dichloromethane and activated carbon, water and the phosphine bases (Triphenylphosphine). The process of disclosure does not need such additives.

Isomerized or branched fatty acids, such as isostearic acid, are currently produced as a secondary product in the dimerization of unsaturated fatty acids. The product is thermal and odor resistant, proving to be great for cosmetic formulations and lubricants. Isostearic acid has also proven to provide oxidation stability for products with long shelf-life requirements. Moreover, the product is known to have an exceptionally low cloud point, making it easily processable. Isostearic acid is more expensive than standard quality fatty acid dimers, and the market of isostearic acid is rapidly expanding. Hence mixtures with high levels of branched fatty acids, and with a little if any fatty acid dimers or oligomers are very relevant. Other side products from processing such as cyclic fatty acids or lactones should be maximally avoided as well.

Until today, branched fatty acids are produced industrially as a by-product of the thermal polymerization of unsaturated fatty acids or fatty acid esters with acid clays as a catalyst. After reaction, a product consisting of a polymeric and a monomeric fraction, is obtained. The polymeric fraction mainly consists of dimers and trimers, while the branched fatty acids can be found in the monomeric fraction. As multiple reactions, e.g., cis trans isomerization, branching, aromatization, double bond shift, hydrogen transfer, occur at the same time, the current reaction product is extremely complex. (1-3)

Current mixtures after processing have monomer fractions that usually vary around 35 wt % as the catalyst primarily forms oligomeric compounds. Multiple purification steps like crystallization and distillation are thus necessary in order to obtain a product consisting primarily of branched fatty acids, which can be considered as a major disadvantage of this process. (4) Since the monomer fraction comprises around 50 wt % branched fatty acids, the overall maximum yield of the process for branched fatty acids is around 17.5 wt %, with the classic process. (5)

Research on the isomerization of unsaturated fatty acids to branched fatty acids has been published in multiple patents and scientific journals. Despite the fact that the use of clay catalysts for the production of branched fatty acids opposes multiple disadvantages, early patents still use clays like Montmorillonite and Bentonite for the isomerization of fatty acids. In these patents, the use of co-catalysts like dichloromethane and activated carbon is described, which should cause a higher yield of branched fatty acids. Despite this, Neuss et al. report mixtures with only 40%, while Foglia et al. report a product with only 57 wt % of branched fatty acids. Moreover, the function of these co-catalysts remains unclear. (6,7)

As the obtained yields of branched fatty acids remain low, researchers have been looking for new catalysts in order to increase the obtained yields. Commercial zeolites are proposed as promising catalysts for the isomerization of unsaturated fatty acids to branched fatty acids. The structure of the zeolite makes it possible to obtain higher yields of branched fatty acids as the pores are too small to form oligomeric side products, but large enough to make diffusion of the branched product possible. Besides this, zeolites have shown to be reusable for multiple times. (8,9)

The oldest patents using zeolites mainly focus on Mordenite and other one dimensional zeolites. It is in one of these patents that the use of water or a small alcohol as an additive is mentioned for the first time. There is assumed that adding water, when the substrate is a fatty acid, or a lower alcohol, when the substrate is a fatty acid ester, suppresses the formation of acid anhydrides by dehydration or dealcoholation of the reagent. (8) However, the use of water or a lower alcohol is countered by another patent published one year later. In this patent, a 68% yield is reached with less catalyst, a lower reaction temperature, in a shorter time period and without adding water. (9)

A few years later, the attention shifts toward other zeolites like Beta. With H-Beta, it is possible to reach conversions up to 74% and to have a product consisting of 46% branched fatty acids. (12)

Thus, there is still a need for an improved process of producing branched fatty acids, particularly a mixture of monobranched and polybranched fatty acids.

Advantageously, the composition of the disclosure is liquid at 0° C. due to polybranched fatty acids and less cyclic compounds. This composition is also stable at high temperatures and resists UV radiation. Advantageously, the composition of the disclosure exhibits better low temperature properties.

Preferably, the cyclic compounds comprise from 14 to 22 carbon atoms, more preferably from 16 to 18 carbon atoms.

Preferably, the cyclic compound content ranges from 0.1% to 5% by weight, more preferably from 3% to 5% by weight, based on the total weight of the composition.

Preferably, cyclic compounds of the composition of the disclosure comprise alicyclic carboxylic acid(s) or ester(s) thereof, which content ranges from 0.1% to 5% by weight based on the total weight of the composition.

Advantageously, the alicyclic carboxylic acid(s) or ester(s) thereof content ranges from 0.1% to 5%, more preferably ranges from 0.1% to 3.5%, still more preferably ranges from 1% to 3.5% by weight, based on the total weight of the composition.

Preferably, the lactone content is less than 5%, more preferably less than 4.5%

### EXAMPLES

### Example 1: ZSM-22

35 grams of fatty acids (comprising 91.3 wt % of oleic acid) and 0.875 grams of H-ZSM-22 (Bonding Chemical) were placed together in a 50 ml Parr autoclave. Air was flushed away three times with nitrogen. A pressure of 7 bar nitrogen was put on the autoclave. While stirring at 600 rpm, the mixture was heated to 250° C. This reaction temperature was held for 4 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction (Table 1)

TABLE 1

| Composition of the hydrogenated | | |
|---|---|---|
| crude reaction mixture | Wt % | |
| Linear C₁₆ fatty acid | 1.1 | |
| Linear C₁₈ fatty acid | 12.4 | |
| Monobranched C₁₈ fatty acids | 57.2 | =71.2 |
| Polybranched C₁₈ fatty acids | 14 | |
| Alicyclic carboxylic acids | 3.7 | |
| Lactones | 4.6 | |
| Oligomers | 6.4 | |
| Others | 0.6 | |
| Mono:multi | 4.1 | |

### Example 2: ZSM-22

35 grams of fatty acids (comprising 91.3 wt % of oleic acid) and 0.875 grams of H-ZSM-22 (Bonding Chemical) were placed together in a 50 ml Parr autoclave. Air was flushed away three times with nitrogen. A pressure of 7 bar nitrogen was put on the autoclave. While stirring at 600 rpm, the mixture was heated to 250° C. This reaction temperature was held for 6 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction (Table 2).

TABLE 2

| | Wt % | |
|---|---|---|
| Linear C₁₆ fatty acid | 1.2 | |
| Linear C₁₈ fatty acid | 8.4 | |
| Monobranched C₁₈ fatty acids | 51.6 | =73.3 |
| Polybranched C₁₈ fatty acids | 21.7 | |
| Alicyclic carboxylic acids | 4.5 | |
| Lactones | 4.2 | |
| Oligomers | 7.7 | |
| Others | 0.7 | |
| Mono:multi | 2.4 | |

### Example 3: Post-Synthetically Treated ZSM-22

35 grams of fatty acids (comprising 90.1 wt % of oleic acid) and 0.875 grams of H-ZSM-22 (Bonding Chemical, post-synthetically treated) were placed together in a 50 ml Parr autoclave. Air was flushed away three times with nitrogen. A pressure of 7 bar nitrogen was put on the autoclave. While stirring at 600 rpm, the mixture was heated to 250° C. This reaction temperature was held for 2 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction (Table 3)

TABLE 3

| Composition of the hydrogenated | | |
|---|---|---|
| crude reaction mixture | Wt % | |
| Linear C₁₆ fatty acid | 1.2 | |
| Linear C₁₈ fatty acid | 11.7 | |
| Monobranched C₁₈ fatty acids | 55.9 | =70.6 |
| Polybranched C₁₈ fatty acids | 16.1 | |
| Alicyclic carboxylic acids | 3.5 | |
| Lactones | 4.1 | |
| Oligomers | 7.1 | |
| Others | 0.4 | |
| Mono:multi | 3.5 | |

### Example 4: ZSM-23

1 gram of granulated H-ZSM-23 (250-500 μm granules) was loaded in a continuous fixed bed reactor. The catalyst bed was heated to 250° C. after which the fatty acids (comprising 87 wt % of oleic acid) were sent over the catalyst bed at a flow rate of 0.05 ml min⁻¹.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction.

| Composition of the | | |
|---|---|---|
| hydrogenated crude | | |
| reaction mixture | Wt % | |
| Linear C₁₆ fatty acid | 3.80 | |
| Linear C₁₈ fatty acid | 9.80 | |
| Monobranched C₁₈ fatty acids | 51.40 | =70.6 |
| Polybranched C₁₈ fatty acids | 19.20 | |
| Acicyclic carboxylic acids | 4.00 | |
| Lactones | 5.00 | |
| Oligomers | 3.90 | |
| Others | 2.90 | |
| Mono:multi | 2.70 | |

### Comparative Example 1: ZSM-5

35 grams of fatty acids (comprising 84.0 wt % of oleic acid) and 2.625 grams of H-ZSM-5 (Zeolyst, CBV2314) were placed together in a 50 ml Parr autoclave. Air was flushed away three times with nitrogen. A pressure of 7 bar nitrogen was put on the autoclave. While stirring at 600 rpm, the mixture was heated to 250° C. This reaction temperature was held for 24 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction. (Table 4)

TABLE 4

| | Wt % | |
|---|---|---|
| Linear C₁₆ fatty acid | 5.4 | |
| Linear C₁₈ fatty acid | 12.7 | |
| Monobranched C₁₈ fatty acids | 32.1 | =60.5 |
| Polybranched C₁₈ fatty acids | 28.4 | |
| Alicyclic carboxylic acids | 3.6 | |
| Lactones | 4.2 | |
| Oligomers | 10.5 | |
| Others | 3.1 | |
| Mono:multi | 1.1 | |

### Comparative Example 2: Post-Synthetically Treated ZSM-5

35 grams of fatty acids (comprising 88.8 wt % of oleic acid) and 0.875 grams of H-ZSM-5 (Zeolyst, CBV2314, post-synthetically treated) were placed together in a 50 ml Parr autoclave. Air was flushed away three times with nitrogen. A pressure of 7 bar nitrogen was put on the autoclave. While stirring at 600 rpm, the mixture was heated to 250° C. This reaction temperature was held for 6 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction. (Table 5)

TABLE 5

| Composition of the hydrogenated | | |
|---|---|---|
| crude reaction mixture | Wt % | |
| Linear C₁₆ fatty acid | 2.3 | |
| Linear C₁₈ fatty acid | 10.9 | |
| Monobranched C₁₈ fatty acids | 29.4 | =62.6 |
| Polybranched C₁₈ fatty acids | 33.2 | |
| Alicyclic carboxylic acids | 6.3 | |
| Lactones | 3.7 | |
| Oligomers | 13.7 | |
| Others | 0.5 | |
| Mono:multi | 0.9 | |

### Comparative Example 3: MOR (Mordenite)

35 grams of fatty acids (comprising 84.0 wt % of oleic acid) and 1.75 grams of H-MOR (Zeolyst, CBV21A) were placed together in a 50 ml Parr autoclave. Air was flushed away three times with nitrogen. A pressure of 7 bar nitrogen was put on the autoclave. While stirring at 600 rpm, the mixture was heated to 250° C. This reaction temperature was held for 8 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction (Table 6).

TABLE 6

| | Wt % | |
|---|---|---|
| Linear C₁₆ fatty acid | 7 | |
| Linear C₁₈ fatty acid | 14 | |
| Monobranched C₁₈ fatty acids | 22.6 | =60.7 |
| Polybranched C₁₈ fatty acids | 38.1 | |
| Alicyclic carboxylic acids | 3.8 | |
| Lactones | 4.7 | |
| Oligomers | 4.5 | |
| Others | 5.3 | |
| Mono:multi | 0.6 | |

### Comparative Example 4: Post-Synthetically Treated MOR

35 grams of fatty acids (comprising 89.4 wt % of oleic acid) and 0.875 grams of H-MOR (Zeolyst, CBV21A, post-synthetically treated) were placed together in a 50 ml Parr autoclave. Air was flushed away three times with nitrogen. A pressure of 7 bar nitrogen was put on the autoclave. While stirring at 600 rpm, the mixture was heated to 250° C. This reaction temperature was held for 6 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction (Table 7).

TABLE 7

| | Wt % | |
|---|---|---|
| Linear C₁₆ fatty acid | 1.8 | |
| Linear C₁₈ fatty acid | 15.9 | |
| Monobranced C₁₈ fatty acids | 23.3 | =60.0 |
| Polybranched C₁₈ fatty acids | 36.7 | |
| Alicyclic carboxilyc acids | 4.9 | |
| Lactones | 5.5 | |
| Oligomers | 9 | |
| Others | 2.9 | |
| Mono:multi | 0.6 | |

### Comparative Example 5: FER+H₂O (Ferrierite)

20 grams of fatty acids (comprising 83.3 wt % of oleic acid), 1 gram of H-FER (Tosoh, 720NHA) and 0.4 grams of distilled water were placed together in a 50 ml Parr autoclave. Air was flushed away three times with nitrogen. A pressure of 7 bar nitrogen was put on the autoclave. While stirring at 600 rpm, the mixture was heated to 260° C. This reaction temperature was held for 6 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction (Table 8).

TABLE 8

| | Wt % | |
|---|---|---|
| Linear C₁₆ fatty acid | 3.2 | |
| Linear C₁₈ fatty acid | 10.1 | |
| Monobranched C₁₈ fatty acids | 53.1 | =65.2 |
| Polybranched C₁₈ fatty acids | 12.1 | |
| Alicyclic carboxylic acids | 3.1 | |
| Lactones | 4.1 | |
| Oligomers | 12.2 | |
| Others | 2.1 | |
| Mono:multi | 4.4 | |

### Comparative Example 5 FER+H₂O+TPP

20 grams of fatty acids (comprising 83.3 wt % of oleic acid), 1 gram of H-FER (Tosoh, 720NHA), 0.4 grams of distilled water and 0.075 g grams of triphenylphosphine were placed together in a 50 ml Parr autoclave. Air was flushed away three times with nitrogen. A pressure of 7 bar nitrogen was put on the autoclave. While stirring at 600 rpm, the mixture was heated to 260° C. This reaction temperature was held for 6 hours.

The reaction mixture was then cooled down to room temperature. Gaseous components were vented away.

A hydrogenation step was conducted on the crude reaction mixture with a 5% of palladium on carbon catalyst. The product was hydrogenated for 6 hours at 80° C. and 20 bar hydrogen pressure.

To characterize the composition of the hydrogenated crude reaction mixture, the latter was esterified with methanol and subsequently analyzed with gas chromatography. A GPC analysis on the hydrogenated crude reaction mixture was performed to quantify the oligomeric fraction (Table 9).

TABLE 9

| | Wt % | |
|---|---|---|
| Linear C₁₆ fatty acid | 2.8 | |
| Linear C₁₈ fatty acid | 25.5 | |
| Monobranched C₁₈ fatty acids | 58.7 | =59.2 |
| Polybranched C₁₈ fatty acids | 0.5 | |
| Alicyclic carboxylic acids | 0.5 | |
| Lactones | 5.3 | |
| Oligomers | 3.5 | |
| Others | 3.2 | |
| Mono:multi | 117.4 | |

By using an inventive process of heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst and in the absence of a Lewis base it was possible to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids.

The disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst and in the absence of a Lewis base.

In another aspect, the disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst, characterized in that the catalyst has 10-ring linear channels without intersecting channels, and in the absence of a Lewis base.

In another aspect, the disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst, characterized in that the catalyst has 10-ring linear channels without interconnecting channels, which otherwise creates intersection spaces of more than 6.2 angstroms, and in the absence of a Lewis base.

In another aspect, the disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst, characterized in that the catalyst is an orthorhombic 10-membered ring (10MR) zeolite composed of 5-, 6-, and 10-rings whereby 10-ring channels (with 10-membered ring openings) are linear unidirectional and one-dimensional, and in the absence of a Lewis base.

In another aspect, the disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst, characterized in that the catalyst has pore/channel openings under 7.5 angstroms, and in the absence of a Lewis base.

In another aspect, the disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst, characterized in that the 10-ring linear channels of the catalyst have a pore diameter of 0.44 nm-0.56 nm×0.51 nm-0.59 nm, preferably of 0.45 nm-0.47 nm×0.52 nm-0.58 nm, and in the absence of a Lewis base.

In another aspect, the disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst, characterized in that the 10-ring linear channels have of the catalyst have openings that are in the range of 5.5-5.9×4.4-4.7 angstroms and preferably 5.6-5.8×4.5-4.7 angstroms and most preferably about 5.7×4.6 angstroms, and in the absence of a Lewis base.

In another aspect, the disclosure provides a way to obtain a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst, characterized in that the catalyst is a zeolite of the group of a ZSM-22 zeolite with TON topology, ZSM-23 zeolite with MTT topology or a ZSM-23/ZSM-22 with MTT (ZSM-23) and TON (ZSM-22) frameworks, and in the absence of a Lewis base.

This disclosure accordingly provides the advantage that composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids can be obtained by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst without a catalyst having a mesoporous crystalline phase.

This disclosure accordingly provides the advantage that composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids can be obtained by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst without a catalyst having a mesoporous crystalline phase a metal containing aluminosilicate catalyst or without adding a metal.

This disclosure accordingly provides the advantage that composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids can be obtained by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst but in the absence of a catalyst containing transition metals, post transition metals, Ln series elements, or an element of the group consisting of B, Ti, Ga, Zr, Ge, Va, Cr, Sb, Nb, and Y.

This disclosure accordingly provides the advantage that a composition comprising branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition or to obtain branched C10-C24 fatty acids with 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the fatty acids can be obtained by heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of a microporous aluminosilicate catalyst but in the absence of an additive or catalyst selected of the group consisting of dichloromethane, activated carbon, water or light alcohols (methanol, ethanol), the Lewis base catalyst triphenylphosphine, the Lewis base catalyst triethylenediamine, a combination of Lewis base catalyst triphenylphosphine, the Lewis base catalyst triethylenediamine and metalloaluminophosphate molecular sieves.

In one embodiment of the disclosure, the invented composition, is the reaction product of (i) isomerizing the linear monoethylenically unsaturated C10-C24 fatty acid(s) from the starting material op disclosure (comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s)), by heating in the presence of the catalyst of disclosure, as described here above, (ii) separating the monomeric fraction from the oligomeric fraction formed in step (i) and (iii) purifying the monomeric fraction to obtain the composition of branched C10-C24 fatty acids.

This disclosure accordingly provides the advantage that by simply heating a starting material comprising at least 80% by weight of linear monoethylenically unsaturated C10-C24 fatty acid(s), or essentially consisting of at least 80% by weight of linear monoethylenically unsaturated C10-C24 fatty acid(s) or consisting of at least 80% by weight of linear monoethylenically unsaturated C10-C24 fatty acid(s) in the presence of the microporous aluminosilicate catalyst and in the absence of other additives, as described here above, a composition of branched C10-C24 fatty acids or esters thereof, comprising 1) at least 70% by weight of mono and polybranched C10-C24 fatty acids or esters thereof and 2) a ratio of monobranched/polybranched C10-24 fatty acids or esters thereof smaller than 5:1 by weight based on the total weight of the composition can be obtained.

In an advantageous embodiment, the composition according to the disclosure and being the reaction product of the process of disclosure further comprises a

In an advantageous embodiment, the composition according to the disclosure and being the reaction product of the process of disclosure further comprises that the ratio monobranched/polybranched C10-24 fatty acids or esters thereof ranges from 1.5:1 to 5:1 by weight based on the total weight of the composition.

In an advantageous embodiment, the composition according to the disclosure and being the reaction product of the process of disclosure further comprises the amount of monobranched C10-C24 fatty acids or esters thereof is at least 45% by weight based on the total weight of the composition.

In an advantageous embodiment, the composition according to the disclosure and being the reaction product of the process of disclosure further comprises the amount of polybranched C10-C24 fatty acids or esters thereof ranges from 0.1 to 30% by weight based on the total weight of the composition.

In an advantageous embodiment, the composition according to the disclosure and being the reaction product of the process of disclosure further comprises the amount of cyclic fatty acids ranges from 0.1 to 5% by weight based on the total weight of the composition.

In an advantageous embodiment, the composition according to the disclosure and being the reaction product of the process of disclosure further comprises the cyclic compounds comprise alicyclic carboxylic acid(s) or ester(s) thereof, which content ranges from 0.1 to 5% by weight based on the total weight of the composition.

In an advantageous embodiment, the composition according to the disclosure and being the reaction product of the process of disclosure further comprises that the amount of linear and branched lactones ranges from 0.1 to 5% by weight based on the total weight of the composition.

In an advantageous embodiment, the composition according to the disclosure and being the reaction product of the process of disclosure further comprises that the amount of oligomers ranges from 0.1 to 8.5% by weight based on the total weight of the composition.

In an advantageous embodiment, the composition according to the disclosure and being the reaction product of the process of disclosure further comprises that the acid value is higher than 165 mg KOH/g.

In an advantageous embodiment, the composition according to the disclosure and being the reaction product of the process of disclosure further comprises that the composition further comprising 1) at least 45% by weight of monobranched C10-C24 fatty acids or esters thereof, 2) 0.1 to 30% by weight of polybranched C10-C24 fatty acids or esters thereof, 3) 0.1 to 5% by weight of cyclic compounds, 6) 0.1 to 5% by weight of linear and branched lactones, 4) 0.1 to 8.5% by weight of oligomers and 5) an acid value higher than 165 mg KOH/g by weight based on the total weight of the composition.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

### REFERENCES TO THE DISCLOSURE

1. Koster R. Solid acid catalyzed conversions of oleochemicals. 2003.
2. Gundstone F D, Harwood J L, Dijkstra A J, eds. The Lipid Handbook. Third edit; 2007. doi:10.1016/0144-8617(88)90020-3.
3. Breuer T E. Dimer acids. In: Kirk-Othmer Encyclopedia of Chemical Technology; 2000.
4. Ngo H L, Hoh E, Foglia T A. Improved synthesis and characterization of saturated branched-chain fatty acid isomers. Eur J Lipid Sci Technol. 2012; 114:213-221. doi:10.1002/ejlt.201000471.
5. Ngo H L, Nuñez A, Lin W, Foglia T A. Zeolite-catalyzed isomerization of oleic acid to branched-chain isomers. Eur J Lipid Sci Technol. 2007; 108:214-224. doi:10.1002/ejlt.200600246.
6. Neuss M, Eierdanz H. U.S. Pat. No. 5,364,949. 1994.
7. Foglia T A, Perlstein T, Nakano Y, Maerker G. U.S. Pat. No. 4,371,469. 1983.
8. Tomifuji T, Abe H, Matsumura Y, Sakuma Y. EP 0 683 150 B1. 1995.
9. Hodgson W R, Lok C M, Roberts G. EP 0 774 451 B2. 1996.
10. Zhang S, Zhang Z, Steichen D. US 2003/100780 A1. 2003.
11. Kenneally C J, Connor D S. EP 1 268 386 B1. 2001.
12. Zhang Z, Zhang S, Steichen D. U.S. Pat. No. 6,831,184 B2. 2004.

1.-23. (canceled)

24. A process for preparing a composition comprising branched C₁₀-C₂₄ fatty acids with
a) at least 70% by weight of mono and polybranched C₁₀-C₂₄ fatty acids or esters thereof,
b) a ratio of monobranched/polybranched C₁₀-C₂₄ fatty acids or esters thereof smaller than 5:1 by weight based upon the total weight of the composition, and
c) an amount of oligomers ranging from only 0.1 to 8.5% by weight based upon the total weight of the composition, wherein a starting material comprising at least 80% by weight of linear monoethylenically unsaturated C₁₀-C₂₄ fatty acid(s), based upon the total weight of the starting material is heated in the presence of a zeolite catalyst, which has 10-ring linear channels without intersecting channels.

25. The process of claim 24, wherein the catalyst is a zeolite selected from the group consisting of a ZSM-22 zeolite with TON topology, ZSM-23 zeolite with MTT topology or a ZSM-23/ZSM-22 with MTT (ZSM-23) and TON (ZSM-22) frameworks.

26. The process of claim 24, wherein the catalyst has 10-ring linear channels without interconnecting channels, which otherwise creates intersection spaces of more than 6.2 angstroms.

27. The process of claim 24, wherein the catalyst is an orthorhombic 10-membered ring (10MR) zeolite composed of 5-, 6-, and 10-rings wherein 10-ring channels (with 10-membered ring openings) are linear unidirectional and one-dimensional.

28. The process of claim 24, wherein the catalyst has pore/channel openings under 7.5 angstroms.

29. The process of claim 24, wherein the 10-ring linear channels of the catalyst have a pore diameter of 0.44 nm-0.56 nm×0.51 nm-0.59 nm.

30. The process of claim 24, wherein the 10-ring linear channels have of the catalyst have openings that are in the range of 5.5-5.9×4.4-4.7 angstroms.

31. The process of claim 24, wherein the starting material is heated in the absence of a catalyst having a mesoporous crystalline phase.

32. The process of claim 24, wherein the starting material is heated in the absence of a metal containing catalyst.

33. The process of claim 24, wherein the starting material is heated in the absence of a catalyst containing transition metals, post transition metals, Ln series elements, or an element of the group consisting of B, Ti, Ga, Zr, Ge, Va, Cr, Sb, Nb, and Y.

34. The process of claim 24, wherein the starting material is heated in the absence of an additive or catalyst selected from the group consisting of dichloromethane, activated carbon, water or light alcohols (methanol, ethanol), the Lewis base catalyst triphenylphosphine, the Lewis base catalyst triethylenediamine, a combination of Lewis base catalyst triphenylphosphine, the Lewis base catalyst triethylenediamine and metalloaluminophosphate molecular sieves.

35. The process of claim 24, wherein (i) isomerizing the linear monoethylenically unsaturated C₁₀-C₂₄ fatty acid(s) from the starting material, by heating in the presence of the catalyst (ii) separating the monomeric fraction from the oligomeric fraction formed in step (i) and (iii) purifying the monomeric fraction to obtain the composition of branched C₁₀-C₂₄ fatty acids.

36. The process of claim 24 that produces a composition of branched C₁₀-C₂₄ fatty acids or esters thereof, the composition comprising:
at least 70% by weight of mono and polybranched C₁₀-C₂₄ fatty acids or esters thereof and
a ratio of monobranched/polybranched C₁₀₋₂₄ fatty acids or esters thereof smaller than 5:1 by weight based upon the total weight of the composition.

37. The process of claim 24 that produces a composition wherein the ratio monobranched/polybranched C₁₀-C₂₄ fatty acids or esters thereof ranges from 1.5:1 to 5:1 by weight based upon the total weight of the composition.

38. The process of claim 24 that produces a composition wherein the amount of monobranched C₁₀-C₂₄ fatty acids or esters thereof is at least 45% by weight based upon the composition's total weight.

39. The process of claim 24 that produces a composition wherein the amount of polybranched C₁₀-C₂₄ fatty acids or esters thereof ranges from 0.1 to 30% by weight based upon the composition's total weight.

40. The process of claim 24 that produces a composition wherein the amount of cyclic fatty acids ranges from 0.1 to 5% by weight based upon the composition's total weight.

41. The process of claim 24 that produces a composition wherein cyclic compounds comprise alicyclic carboxylic acid(s) or ester(s) thereof, which content ranges from 0.1 to 5% by weight based upon the composition's total weight.

42. The process of claim 24 that produces a composition wherein the amount of linear and branched lactones ranges from 0.1 to 5% by weight based upon the composition's total weight.

43. The process of claim 24 that produces a composition wherein the acid value is higher than 165 mg KOH/g.

44. The process of claim 24 that produces a composition further comprising:
at least 45% by weight of monobranched C₁₀-C₂₄ fatty acids or esters thereof,
0.1 to 30% by weight of polybranched C₁₀-C₂₄ fatty acids or esters thereof,
0.1 to 5% by weight of cyclic compounds,
0.1 to 5% by weight of linear and branched lactones,
0.1 to 8.5% by weight of oligomers, and
an acid value higher than 165 mg KOH/g by weight based upon the composition's total weight.
